(19) United States
(12) Patent Application Publication
(10) Pub. No.: US 2022/0067841 A1
(43) Pub. Date: Mar. 3, 2022

(54) METHOD AND SYSTEM FOR PROCURING HEALTHCARE SERVICES

(71) Applicant: Transparent Health Marketplace, Inc., Wayne, PA (US)

(72) Inventor: Eric Hanson, Bumpass, VA (US)

(73) Assignee: Transparent Health Marketplace, Inc., Wayne, PA (US)

(21) Appl. No.: 17/467,077

(22) Filed: Sep. 3, 2021

Related U.S. Application Data
(60) Provisional application No. 63/074,336, filed on Sep. 3, 2020.

(51) Int. Cl. G06Q 40/08; G16H 40/20; G06Q 30/08

(52) U.S. Cl. CPC G06Q 40/08; G06Q 30/08; G16H 40/20

(57) ABSTRACT

Techniques are provided for procuring healthcare services. A request to procure healthcare services for a patient is obtained. Provider subscription profiles corresponding to healthcare providers are generated based on subscriber information and provider subscription criteria. Bid auction invitations are generated by assigning a bid invitation ranking to each provider subscription profile and selecting healthcare providers to participate in a bid auction based on the bid invitation rankings. A pre-bill claim form is generated based on at least one bill template related to one or more medical classification codes related to the healthcare services. An auction engine is configured to conduct a bid auction among the selected healthcare providers, which results in a selection of a healthcare provider for the healthcare services and, upon confirmation that the healthcare services have been provided, a computing device or network platform is configured to take an action based on the pre-bill claim form.

### TECHNICAL FIELD

This disclosure relates generally to procuring healthcare services, and more specifically to inventive methods of facilitating, with the assistance of a computer system, transactions between payors, healthcare providers and patients for procuring healthcare services.

### BACKGROUND

Current models for transacting healthcare services have many known static and non-competitive inefficiencies. For example, manual processes and expensive middlemen are characteristic of current healthcare marketplaces.

Ideally, more sensible healthcare procurement methods would utilize modern marketplace technologies, such as those currently employed in travel, finance, and other industries, to bring much-needed efficiency and transparency to the relationship between healthcare payors, providers, and patients. More efficient methods would automate costly manual processes and eliminate expensive middlemen to drive payor cost savings. Particularly, payors of healthcare services (e.g., insurance carriers, third-party administrators, adjusters, employers, individual consumers, etc.) would be enabled to create their own dynamic marketplace of healthcare providers with an expressed interest in competing for their business. Meanwhile, healthcare providers (e.g., hospitals, clinics, laboratories, etc.) would have opportunities to access scripts from a variety of sources and the ability to control price, optimize resources and maximize revenue by filling more available appointments and moving excess inventory. The transparency of the interrelationship between healthcare payors, providers, and patients would include consistent use of provider data points that drive the value of the offering of healthcare services and the ultimate timing, selection, and reimbursement rate of the healthcare providers.

Unfortunately, numerous technical hurdles have confined more innovative methods and systems for procuring healthcare services to only limited applications and the subject of further research. Technical development has been lacking for the several automated processes that would be required to eliminate burdensome steps that, at present, only can be performed manually. For example, many current healthcare procurement platforms are insufficient for determining which healthcare providers are to be included in an open bidding marketplace auction for healthcare services, and do not structure bid auctions around cost transparency and the impartial invitation and selection of healthcare providers. Therefore, it is currently difficult for healthcare providers to identify and bid on new patient scripts from multiple payor sources in real-time. Further, many current healthcare procurement platforms often do not employ methods for facilitating the expedited billing for services, which disadvantages all parties involved. Moreover, current platforms often lack the data intelligence technology and other security precautions required for the protection of payors, healthcare providers and patients.

Therefore, until now, there has remained a clear need for new, automated methods of efficiently and transparently procuring healthcare services for the benefit of payors, healthcare providers and patients.

### SUMMARY

Computer-implemented systems, methods, and articles of manufacture for procuring healthcare services are described herein. The various embodiments may be implemented using a web-based transactional platform that can automatically process various requests related to the procurement of healthcare services, including workers' compensation-related healthcare services for the treatment of patients. Particularly, a workflow transaction engine, executable on a processor according to software instructions stored in a memory device, is operable to automatically perform various operations related to the conversion of user authorization input, price comparisons, bid processes, service confirmations, and the generation of billing data. The operations include automated methods for determining the healthcare providers to be included in an automated open bidding marketplace auction for healthcare services. The operations also include automated methods for generating a “pre-bill” claim form for healthcare services from bill templates using medical classification codes, such as Current Procedural Terminology® (CPT®) codes, Health Care Common Procedural Coding System (HCPCS) codes, pharmaceutical National Drug Codes (NDCs), Diagnostic and Statistical Manual (DSM) psychiatric/mental disorder codes, Current Dental Terminology (CDT) codes, or other standard nomenclatures used for healthcare billing. The generated pre-bill claim form may be used to generate provider bid invitations and serve as an individual bid contract.

In one embodiment, a request to procure healthcare services for a patient is obtained. The patient may be an employee and the healthcare services may be workers' compensation healthcare services. The request may comprise information regarding the patient's availability including at least one of a time and date parameter, and geographical information related to the patient including a radius around a geographical location or a distance from an address or location of the patient. A provider subscription profile corresponding to each of the plurality of healthcare providers is generated based on subscriber information and provider subscription criteria, which may be related to workers' compensation healthcare services. Bid auction invitations are generated by processing the provider subscription profiles using a selection algorithm operable to assign a bid invitation ranking to each provider subscription profile based on the request and ranking parameters, and select healthcare providers from among the plurality of healthcare providers to participate in a bid auction based on the bid invitation rankings. A pre-bill claim form is generated based on at least one bill template related to one or more medical classification codes related to the healthcare services. The pre-bill claim form may be a CMS-1500 claim form or a successor CMS claim form, and standard nomenclature codes such as CPT®, HCPCS, and International Classification of Diseases, Tenth Revision, Clinical Modification (ICD-10-CM) codes or their successors may be used. An auction engine is instantiated to automatically conduct a bid auction among the selected healthcare providers based on the bid auction invitations and the one or more medical classification codes, where the bid auction results in a selection of at least one of the plurality of healthcare providers to provide the healthcare services. Upon confirmation that the healthcare services have been provided by the at least one of the plurality of healthcare providers, a computing device or network platform is configured to take an action based on the pre-bill claim form.

In some embodiments, the provider subscription criteria may include parameters based on one or more of the following: provider demographic information, provider financial information, provider location information, provider practice information, provider procedural codes, and provider pricing information.

In some embodiments, the provider subscription profiles may include provider data related to one or more of the following: demographic information, financial information, location information, practice information, procedural codes, and pricing information.

In some embodiments, the request may comprise information regarding the patient's availability including at least one of a time and date parameter, geographical information related to the patient that may include a radius around a geographical location or a distance from an address or location of the patient.

In some embodiments, the pre-bill claim form may be generated using a statistical or machine learning technique including one or more of a data mining, predictive modelling, and deep learning technique.

In some embodiments, the bid auction invitations may comprise the pre-bill claim form, and the pre-bill claim form may comprise an individual bid contract.

In some embodiments, the ranking parameters may be derived from the provider information based on one or more of the following: services provided, location, government regulations, client/payor preferences, auction configuration parameters, scheduling availability, quality metrics, and behavior metrics.

In some embodiments, the ranking parameters may be derived using a statistical or machine learning technique including one or more of a data mining, predictive modelling, and deep learning technique, and a selection model may be trained using the provider subscription profiles corresponding to the one or more healthcare providers.

In some embodiments, the auction engine may be configured using the trained selection model to automatically conduct the auction among the selected healthcare providers.

In some embodiments, the action may comprise one or more of generating an invoice to a payor for the healthcare services, generating an alert notification, and automatically initiating an Electronic Funds Transfer (EFT), Automated Clearing House (ACH) or other electronic payment method.

Various objects, features, aspects and advantages of the inventive subject matter will become more apparent from the following specification, along with the accompanying drawings in which like numerals represent like components.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a block diagram of a system for procuring healthcare services in accordance with an embodiment.

FIG. 2 illustrates a block diagram of a platform for procuring healthcare services in accordance with an embodiment.

FIG. 3 illustrates a flow diagram of example operations for procuring healthcare services in accordance with an embodiment.

FIG. 4 illustrates a flow diagram of example operations for generating a pre-bill claim form in accordance with an embodiment.

FIG. 5A illustrates a flow diagram of example operations for selecting healthcare providers from among the plurality of healthcare providers to participate in a bid auction in accordance with an embodiment.

FIG. 5B illustrates a flow diagram of example operations for automatically conducting a bid auction among healthcare providers selected to participate in accordance with an embodiment.

FIG. 6 illustrates a flow diagram of example operations for generating an invoice for the healthcare services performed in accordance with an embodiment.

FIG. 7 illustrates a flow diagram of example operations for invoicing a payor for the healthcare services performed in accordance with an embodiment.

FIG. 8 illustrates a block diagram of an exemplary client-server relationship that can be used for implementing one or more aspects of the various embodiments; and

FIG. 9 illustrates a block diagram of a distributed computer system that can be used for implementing one or more aspects of the various embodiments.

While the invention is described with reference to the above drawings, the drawings are intended to be illustrative, and other embodiments are consistent with the spirit, and within the scope, of the invention.

### DETAILED DESCRIPTION

The various embodiments now will be described more fully hereinafter with reference to the accompanying drawings, which form a part hereof, and which show, by way of illustration, specific examples of practicing the embodiments. This specification may, however, be embodied in many different forms and should not be construed as being limited to the embodiments set forth herein; rather, these embodiments are provided so that this specification will be thorough and complete, and will fully convey the scope of the invention to those skilled in the art. Among other things, this specification may be embodied as methods or devices. Accordingly, any of the various embodiments herein may take the form of an entirely hardware embodiment, an entirely software embodiment or an embodiment combining software and hardware aspects. The following specification is, therefore, not to be taken in a limiting sense.

Throughout the specification and claims, the following terms take the meanings explicitly associated herein, unless the context clearly dictates otherwise:

The phrase “in one embodiment” as used herein does not necessarily refer to the same embodiment, though it may. Thus, as described below, various embodiments of the invention may be readily combined, without departing from the scope or spirit of the invention.

As used herein, the term “or” is an inclusive “or” operator and is equivalent to the term “and/or,” unless the context clearly dictates otherwise.

The term “based on” is not exclusive and allows for being based on additional factors not described unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Within the context of a networked environment where two or more components or devices are able to exchange data, the terms “coupled to” and “coupled with” are also used to mean “communicatively coupled with”, possibly via one or more intermediary devices.

In addition, throughout the specification, the meaning of “a”, “an”, and “the” includes plural references, and the meaning of “in” includes “in” and “on”.

Although some of the various embodiments presented herein constitute a single combination of inventive elements, it should be appreciated that the inventive subject matter is considered to include all possible combinations of the disclosed elements. As such, if one embodiment comprises elements A, B, and C, and another embodiment comprises elements B and D, then the inventive subject matter is also considered to include other remaining combinations of A, B, C, or D, even if not explicitly discussed herein. Further, the transitional term “comprising” means to have as parts or members, or to be those parts or members. As used herein, the transitional term “comprising” is inclusive or open-ended and does not exclude additional, unrecited elements or method steps.

Throughout the following disclosure, numerous references may be made regarding servers, services, interfaces, engines, modules, clients, peers, portals, platforms, or other systems formed from computing devices. It should be appreciated that the use of such terms is deemed to represent one or more computing devices having at least one processor (e.g., ASIC, FPGA, DSP, x86, ARM, ColdFire, GPU, multi-core processors, etc.) configured to execute software instructions stored on a computer readable tangible, non-transitory medium (e.g., hard drive, solid state drive, RAM, flash, ROM, etc.). For example, a server can include one or more computers operating as a web server, database server, or other type of computer server in a manner to fulfill described roles, responsibilities, or functions. One should further appreciate the disclosed computer-based algorithms, processes, methods, or other types of instruction sets can be embodied as a computer program product comprising a non-transitory, tangible computer readable medium storing the instructions that cause a processor, when instantiated, to execute the disclosed steps. The various servers, systems, databases, or interfaces can exchange data using standardized protocols or algorithms, possibly based on HTTP, HTTPS, AES, public-private key exchanges, web service APIs, known financial transaction protocols, or other electronic information exchanging methods. Data exchanges can be conducted over a packet-switched network, a circuit-switched network, the Internet, LAN, WAN, VPN, or other type of network.

As used in the description herein and throughout the claims that follow, when a system, engine, server, device, module, or other computing element is described as being configured to perform or execute functions on data in a memory, the meaning of “configured to” or “programmed to” is defined as one or more processors or cores of the computing element being programmed by a set of software instructions stored in the memory of the computing element to execute the set of functions on target data or data objects stored in the memory.

It should be noted that any language directed to a computer should be read to include any suitable combination of computing devices or network platforms, including servers, interfaces, systems, databases, agents, peers, engines, controllers, modules, or other types of computing devices operating individually or collectively. One should appreciate the computing devices comprise a processor configured to execute software instructions stored on a tangible, non-transitory computer readable storage medium (e.g., hard drive, FPGA, PLA, solid state drive, RAM, flash, ROM, etc.). The software instructions configure or program the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps associated with implementations of computer-based algorithms, processes, methods, or other instructions. In some embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

A focus of the disclosed inventive subject matter is to enable construction, configuration, and operational improvement of a distributed computer-based platform capable of operating on vast quantities of digital data beyond the capabilities of a human for purposes including managing the procurement of healthcare services. As used herein, healthcare services may relate to, for example, diagnostics, durable medical equipment, home health, physical medicine, translation, transportation, hospital care, ambulatory surgery care or other forms of facility care, pharmaceutical services, pharmacy prescription services, psychiatric/mental health services, dental services, and other related activities.

One should appreciate that the disclosed techniques provide many advantageous technical effects including automated methods for determining the healthcare providers to be included in an automated open bidding marketplace auction for healthcare services. The techniques described herein employ logic to automate and improve processes currently performed using manual human effort. For example, the techniques include automated methods for generating a pre-bill claim form for healthcare services using at least one billing template related to one or more medical classification codes, such as CPT® codes or ICD-10-CM codes, and automated methods for conducting a bid auction for healthcare services. Further, the disclosed techniques have been designed to support provider data accuracy and allow for processing data algorithms and complex permutations on a scale and speed not seen in typical healthcare marketplace platforms.

It should also be appreciated that the following specification is not intended as an extensive overview, and as such, concepts may be simplified in the interests of clarity and brevity.

Computer-implemented systems, methods, and articles of manufacture for procuring healthcare services are described herein. In the various embodiments, automated processes connect healthcare service providers with carriers, third-party administrators, employers responsible for helping patients (e.g., injured employees), and/or patients acting as direct consumers of healthcare to receive appropriate healthcare services. The techniques herein automate various previously manual processes, including processes for determining which healthcare providers are to be included in an open bidding marketplace auction for healthcare services, for conducting a bid auction for healthcare services, and for generating a pre-bill claim form for healthcare services using billing templates related to medical classification codes.

For healthcare providers (e.g., doctors, retail pharmacies, mail order pharmacies, specialty providers, hospitals, clinics, laboratories, etc.), the inventive subject matter enables access to scripts from payors and the ability to control price, optimize resources and maximize revenue by filling more available appointments and moving excess inventory. For example, the systems and methods described herein enable real-time bidding by healthcare providers as requests for proposals for healthcare services, e.g., healthcare services requested for injured workers, are obtained. Using techniques described herein, qualified healthcare providers are selected to participate in a bid auction to provide such services based on a plurality of parameters including, for example, their services offered and their proximity to the patient. The healthcare providers selected to participate are then enabled to respond with a best price offering to be awarded the script for healthcare services. In some embodiments, healthcare providers are enabled to establish a “Buy it Now” offer price which payors can select immediately to procure healthcare services for patients. Further, the systems and methods described herein enable healthcare providers to search for opportunities to submit additional price proposals. This approach provides healthcare providers with access to service opportunities that they would not otherwise have access to under conventional methods. In some embodiments, the approach provides opportunities for healthcare providers who wish to fill excess capacity. For example, if a patient cancels a service, a healthcare provider using systems and methods described herein can search for opportunities to replace that opening with another service opportunity.

For payors of healthcare services (e.g., insurance carriers, third-party administrators, adjusters, employers, clinical management, etc.), the inventive subject matter enables a dynamic computer network-based marketplace of healthcare providers with an expressed interest in competing for their business, without being a conventionally organized healthcare network that holds static contracts with healthcare providers. Instead, the inventive subject matter enables payors to define a “best value” for healthcare services using selected criteria (e.g., price, performance, availability, distance, etc.), and then facilitates a spot-bidding auction to connect winning healthcare providers with payors for each script. Thus, the inventive subject matter minimizes the administrative burden and cost of coordinating healthcare services while reducing the risk of having to pay more than market price for such services.

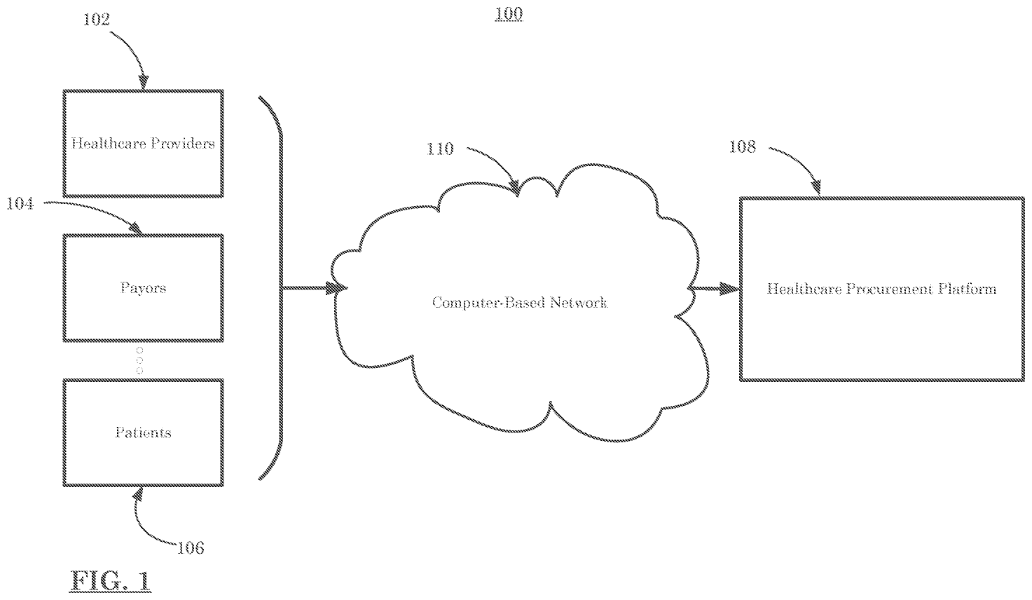

FIG. 1 illustrates a block diagram of a system for procuring healthcare services in accordance with an embodiment. The diagram depicts the system participants and interactions between the participants. In one exemplary embodiment, the techniques described herein for procuring healthcare services can be implemented as a Platform-as-a-Service (PaaS) within a cloud computing environment 100. As illustrated, healthcare providers 102, payors 104, and patients 106 at computing devices (e.g., a personal computer, a laptop computer, a workstation, a mobile communication device such as a wireless phone, a tablet, etc.) with user interfaces (e.g., dashboard-type applications or webpages presented on a display screen for displaying information) can access healthcare procurement platform 108 via computer-based network 110. In some embodiments, patients 106 may have indirect access to services provided via platform 108, e.g., via an insurance carrier or employer. Alternatively, patients 106 may have direct access to services provided via platform 108.

In the exemplary embodiment of FIG. 1, computer-based network 110 is the Internet. In other embodiments, computer-based network 110 may comprise one or more of a number of different types of local or distributed computer-based networks, such as, for example, an intranet, a local area network (LAN), a wide area network (WAN), a wireless network, a Fibre Channel-based storage area network (SAN), or Ethernet. Other computer-based networks may be used. Alternatively, computer-based network 110 may comprise a combination of different types of networks.

Healthcare procurement platform 108 provides healthcare procurement services to healthcare provider 102, payor 104, and patient 106 users via computer-based network 110, enabling an open bidding marketplace auction and pre-billing for healthcare services. Within healthcare procurement platform 108, a plurality of servers, which may be communicatively connected across a distributed network, can host applications for implementing the techniques described herein, such as applications for generating a provider subscription profile; generating a pre-bill claim form, selecting healthcare providers to participate in a bid auction; automatically conducting a bid auction; and configuring a computing device or network platform to take an action based on the pre-bill claim form.

Healthcare procurement platform 108 may be configured to allow for real-time connectivity with multiple healthcare providers 102, payors 104, and patients 106 via computer-based network 110. Thus, healthcare procurement platform 108 is operable to function as a seamless extension of each individual healthcare payor's or provider's claims system and processes, and as a single channel for scripts, scheduling, post-appointment follow-up, automated updates, document/billing management, and various consumer-driven healthcare tasks.

In an embodiment, healthcare procurement platform 108 may comprise applications for implementing a subscriber subscription portal. The subscriber subscription portal may be operable to perform functions related to linking healthcare provider subscribers to the services provided via healthcare procurement platform 108, including, for example, one or more of the following: creating an individual provider subscription including uploaded provider demographic and group information, financial information, individual provider location and practice customization, procedural codes, and pricing guidance; storing provider demographics; indicating provider status/availability to other users, determining providers to be included in a bid auction; distributing bid invitations to selected providers; tracking the status of bid invitations, and allowing for provider search and profile views.

In an embodiment, healthcare procurement platform 108 may comprise one or more applications for implementing a payor portal. For example, the payor portal may be operable to allow for creating a new payor profile/account; updating payor configurations; customizing a payor marketplace; assigning/removing providers to/from a payor marketplace; viewing the status of invoices; and payor management of accounts (e.g., via dashboards, reporting, etc.). The payor portal also may enable payors 104 to edit/view script (i.e., prescription) details, edit/view patient details, add/edit/view/cancel appointments, select a winning provider, set bid award preferences (e.g., limitations on mileage, price, availability, drive time, etc.), set marketplace state(s)/geographic limitations and other options (e.g., specialty types), set fee structures, and (in limited circumstances) manually source providers on scripts.

Healthcare procurement platform 108 also may support a variety of administrative functions. For example, healthcare procurement platform 108 may comprise applications for implementing administrative functions including one or more of: creating/editing/removing users or user groups; adding users to/editing users from user groups; deleting user groups; managing administrator/super administrator access within user groups; adding/assigning/editing/viewing user roles within user groups; functions related to standard pricing and discounts; functions related to value-based care; etc. In an embodiment, healthcare procurement platform 108 may comprise applications to allow administrators access to one or more of the provider portal and payor portal functions described above, including access to payor and provider analytics reports and dashboards.

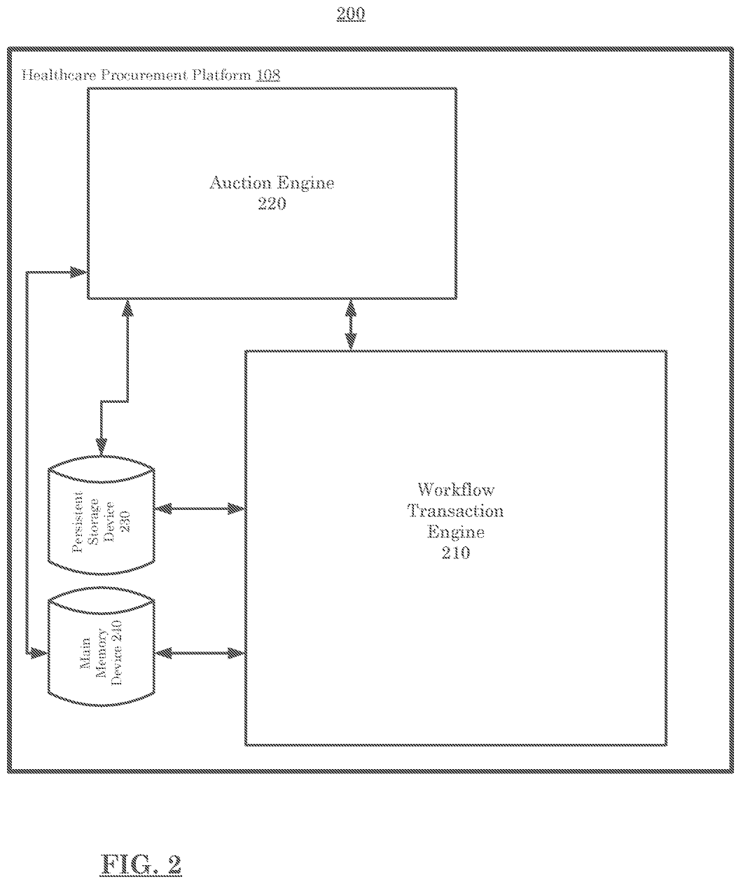

FIG. 2 illustrates a block diagram of a platform for procuring healthcare services in accordance with an embodiment. In block diagram 200, healthcare procurement platform 108 may comprise various localized or distributed elements for procuring healthcare services, including workflow transaction engine 210, auction engine 220, persistent storage device 230, and main memory device 240.

Workflow transaction engine 210 may perform a variety of functions related to, for example, confirmation of services, accounting, dual-sided transaction pricing, clearinghouse, and compliance and stakeholder reporting operations.

In an embodiment, workflow transaction engine 210 may be configured to obtain a request to procure healthcare services for a patient from either one or both of persistent storage device 230 and main memory device 240, and generate a provider subscription profile corresponding to each of the plurality of healthcare providers based on the subscriber information and provider subscription criteria. The workflow transaction engine 210 then may be configured to generate bid auction invitations by processing the provider subscription profiles using a selection algorithm, which may be stored in either one or both of persistent storage device 230 and main memory device 240. When executed by workflow transaction engine 210, the selection algorithm may be operable to assign a bid invitation ranking to each provider subscription profile based on the request and ranking parameters, and select healthcare providers from among the plurality of healthcare providers to participate in a bid auction based on the bid invitation rankings. Workflow transaction engine 210 then may generate a pre-bill claim form based on at least one bill template related to the one or more medical classification codes.

Auction engine 220 may be configured to execute algorithmic functions that utilize both pre-set and adjustable parameters related to, for example, healthcare provider services offered, service location and distance, pricing settings/ranges, quality outcomes (e.g., speed/responsiveness, treatment data, etc.), appointment availability, and payor preference settings and weightings, to select a winning provider and generate a market connection between healthcare payors, providers, and patients.

In an embodiment, auction engine 220 may be instantiated by workflow transaction engine 210 to automatically conduct a bid auction among the selected healthcare providers based on the bid auction invitations and the one or more medical classification codes, where the bid auction results in a selection of at least one of the plurality of healthcare providers to provide the healthcare services. Upon confirmation that the healthcare services have been provided by the at least one of the plurality of healthcare providers, workflow transaction engine 210 may configure and/or instantiate a computing device or network platform to take an action based on the pre-bill claim form. For example, workflow transaction engine 210 may configure and/or instantiate a computing device or network platform based on the pre-bill claim form to, for example, generate an invoice to a payor of the healthcare services; generate an alert notification; automatically initiate an Electronic Funds Transfer (EFT), Automated Clearing House (ACH) or other electronic payment method; create or automatically update a profile/account; create or automatically update payor or provider configurations; set fee structures, standing and dynamic pricing, and/or fee-for-service structures; and automatically allow for access to one or more provider portal and payor portal functions. For example, workflow transaction engine 210 may configure and/or instantiate a computing device or network platform to execute an application that obtains or generates data documenting payor preferences (e.g., time to care, distance, price, quality, treatment protocols, etc.) that may be used as feedback for selection criteria and weighting in a bid auction. In another example, workflow transaction engine 210 may configure and/or instantiate a computing device or network platform to execute an application that obtains or generates healthcare provider profile details such as available locations and service types, standing and dynamic pricing, and fee-for-service structures that may also be used as feedback for selection criteria and weighting in a bid auction.

It should be noted that the elements in FIG. 2, and the various functions attributed to each of the elements, while exemplary, are described as such solely for the purposes of ease of understanding. One skilled in the art will appreciate that one or more of the functions ascribed to the various elements may be performed by any one of the other elements, and/or by an element (not shown) configured to perform a combination of the various functions. Therefore, it should be noted that any language directed to a workflow transaction engine 210, an auction engine 220, a persistent storage device 230 and a main memory device 240 should be read to include any suitable combination of computing devices and/or computer-based network platforms, including servers, interfaces, systems, databases, agents, peers, engines, controllers, modules, or other types of computing devices operating individually or collectively to perform the functions ascribed to the various elements. Further, one skilled in the art will appreciate that one or more of the functions of the platform of FIG. 2 described herein may be performed within the context of a client-server relationship, such as by one or more servers, one or more client devices (e.g., one or more user devices) and/or by a combination of one or more servers and client devices.

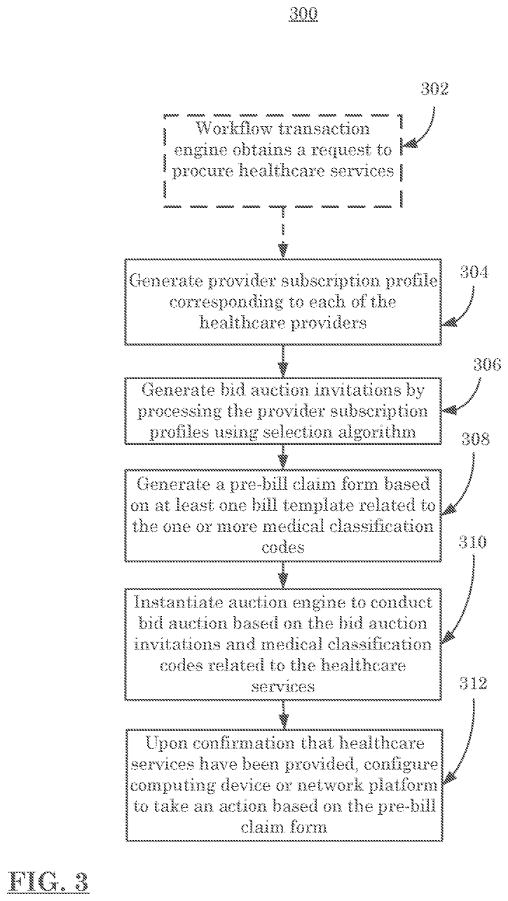

FIG. 3 illustrates a flow diagram of example operations for procuring healthcare services in accordance with an embodiment. For example, one or more of the steps described in FIG. 3 may be performed by at least one processor coupled with a memory (e.g., either one or both of persistent storage device 230 and main memory device 240) operating as a workflow transaction engine (e.g., workflow transaction engine 210) upon the execution of software instructions.

In flow diagram 300, a request to procure healthcare services for a patient is obtained at step 302. For example, the patient may be an employee and the healthcare services requested may be workers' compensation healthcare services. Alternatively, the procurement of healthcare services may be related to managed care plans such as Health Maintenance Organizations (HMO), Preferred Provider Organizations (PPO), or other healthcare groups (e.g., Health Savings Accounts (HSAs), Flexible Spending Accounts (FSAs)) that coordinate the activities of healthcare payors, providers, and patients. In an embodiment, the request may comprise information regarding the patient's availability including at least one of a time and date parameter, and geographical information related to the patient, including a radius around a geographical location or a distance from an address or location of the patient.

At step 304, a provider subscription profile corresponding to each of the plurality of healthcare providers is generated based on subscriber information and provider subscription criteria. For example, the provider subscription profiles may include provider data related to one or more of demographic information, financial information, location information, practice information, procedural codes, and pricing information. The provider subscription criteria may be related to workers' compensation healthcare services, consumer-driven healthcare plans (e.g., HSAs, FSAs), managed care plans, or other group healthcare services. In some embodiments, the provider subscription criteria may include various parameters. For example, the provider subscription criteria may be based on one or more of provider demographic information, provider financial information, provider location information, provider practice information, provider procedural codes, and provider pricing information.

At step 306, bid auction invitations are generated by processing the provider subscription profiles using a selection algorithm operable to assign a bid invitation ranking to each provider subscription profile based on the request and ranking parameters, and select healthcare providers from among the plurality of healthcare providers to participate in a bid auction based on the bid invitation rankings. The ranking parameters may be derived from the provider information based on one or more of the following: services provided, location, government regulations, client/payor preferences, auction configuration parameters, scheduling availability, quality metrics, and behavior metrics. The selection algorithm may utilize derived decision criteria that are evaluated against payor preferences for scoring and selection. For example, past and current data regarding, e.g., payor and or patient/client preferences may be obtained, and derived decision criteria may be generated based on the past and current data. Then, an initial relative weight or score may be determined for each of the payor preferences, and the ranking parameters may be derived from the provider information based on the derived decision criteria.

In an exemplary embodiment, the ranking parameters may be derived using a statistical or machine learning technique including one or more of a data mining, predictive modelling, and deep learning technique. For example, a selection model, e.g., a neural network architecture trained using one or more meta learning algorithms, may be instantiated to determine the ranking parameters, a “next best action”, and/or other “actionable insights” related to selecting healthcare providers to participate in the bid auction. The selection model may be trained using raw or derived data from training provider subscription profiles, such as those corresponding to the one or more healthcare providers, or other raw or derived training data. For example, other types of training data may be derived from data related to providers' proven outcomes or quality of care; a combination of provider, payor, and/or patient data; national and/or regional pricing data, demographic data, and/or other statistical data.

At step 308, a pre-bill claim form is generated based on at least one bill template related to one or more medical classification codes related to the healthcare services. The pre-bill claim form may be, for example, a CMS-1500 claim form (or a successor CMS claim form) that includes CPT® codes or ICD-10-CM codes to describe healthcare services. The pre-bill claim form may be generated based on data derived from one or more medical network, payor, and/or other third-party data sources to determine appropriate and/or most likely treatments calculated against industry regulations or allowances. In some embodiments, the pre-bill claim form may be generated based on the derived data using a statistical or machine learning technique including one or more of a data mining, predictive modelling, and deep learning technique, or one or more bill builder templates. Further, in some embodiments, the pre-bill claim form may be used to generate bid invitations to the healthcare providers selected to participate in the bid auction and may comprise an individual bid contract.

At step 310, an auction engine, e.g., auction engine 220, is instantiated based on the bid auction invitations and the one or more medical classification codes to automatically conduct a bid auction among the selected healthcare providers. For example, the auction engine may be configured to conduct a bid auction based on the bid auction invitations and the one or more medical classification codes by, during run-time operations, continuously recalculating weighted or scored values derived from current and/or historical healthcare provider data and payor selection criteria (e.g., speed to care, location, quality, price, treatment protocols, etc.) until a relative ranking is determined for each of the invited healthcare providers. In an embodiment, the weighted or scored values may be continuously recalculated during run-time operations using one or more of a weighted least squares/weighted linear regression equation, a polynomial weights algorithm, a weighted majority algorithm, a randomized weighted majority algorithm, an iteratively reweighted least squares algorithm, and a prediction based on a neural network model trained using one or more meta learning algorithms. Upon completion of run-time operations (e.g., at the end of a selected time interval), the bid auction results in a selection of at least one of the plurality of healthcare providers to provide the healthcare services. For example, a healthcare provider's spot price contract may be selected based on the amounts bid, or a “Buy it Now” price established by a healthcare provider may be selected. In an exemplary embodiment, the auction engine may be configured using the trained selection model described above to automatically conduct the auction among the selected healthcare providers.

In one illustrative example of how the transparency provided by the operations for procuring healthcare services described herein may occur in a workers' compensation context, a physical therapist provider may submit an initial standing offer of $80 per patient visit. However, due to offers submitted by other physical therapists in the same defined service area, she may need to lower her price to $75 per patient visit to win the course of treatment (e.g., 6-8 visits) for a patient. Therefore, she will know that she will receive $75 per visit if her bid wins, and if she agrees to see the patient within the time specified for the treatment to commence (e.g., on the next business day). While price may be important to a payor of the requested treatment, the payor may also highly value speed to care. Thus, the auction engine determines which provider to select in a bid auction based at least in part on weighted values derived from one or more of the payor's preferred selection criteria (e.g., speed to care may be represented by a weighted value greater than a weighted value representing price). Once a provider is selected upon competition of the bid auction, the payor will be informed of the winning bid price and cost for the entire treatment cycle. Moreover, the patient may benefit from a likely decrease in the time between the request for treatment and the appointment (typically a week or more faster than traditional scheduling methods), and the selected provider may benefit from a reimbursement process enabled to run in a matter of days, rather than the usual 30/60/90-day billing cycle.

Upon confirmation that the healthcare services have been provided by the at least one of the plurality of healthcare providers at step 312, a computing device or network platform is configured and/or instantiated to take an action based on the pre-bill claim form. For example, the action may comprise one or more of generating invoice to a payor of the healthcare services, generating an alert notification, and automatically initiating an Electronic Funds Transfer (EFT), Automated Clearing House (ACH) or other electronic payment method.

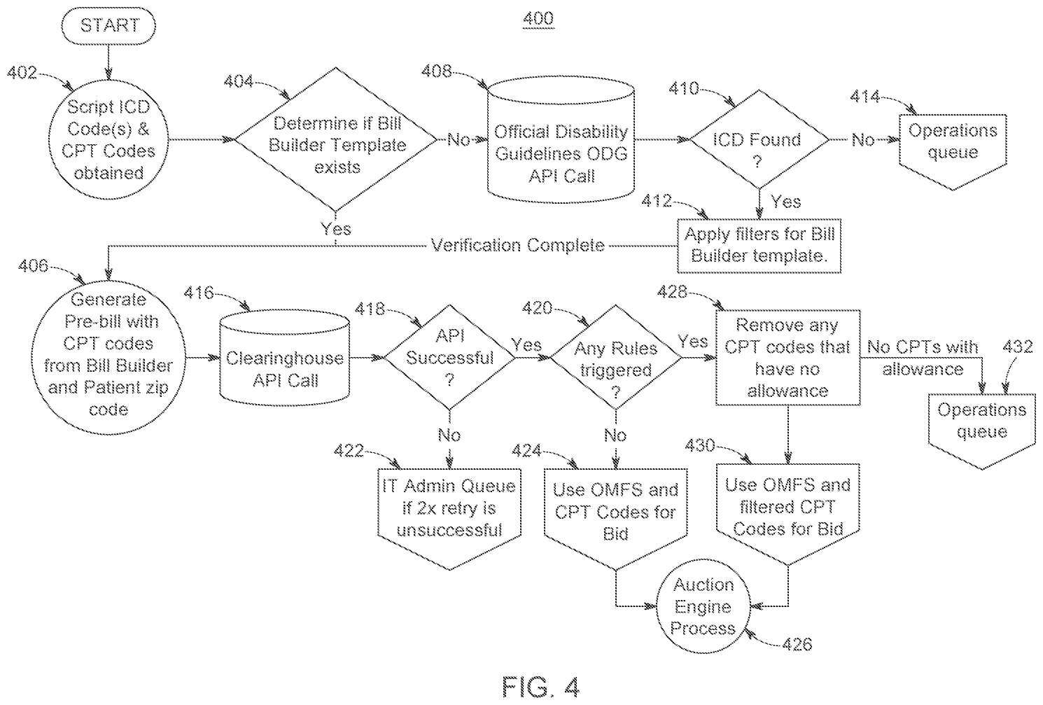

FIG. 4 illustrates a flow diagram of example operations for generating a pre-bill claim form in accordance with an embodiment. The inventive subject matter reverses typical pricing processes by, inter alia, generating a pre-bill claim form for healthcare services using one or more medical classification codes that is visible to payors and potential healthcare providers. The medical classification codes are derived from the American Medical Association's (AMA's) Current Procedure Terminology (CPT®) system and the International Classification of Diseases (ICD) procedure and diagnosis coding systems, or other relevant, widely accepted claim coding nomenclature. Therefore, as generated by the various operations described above, the pre-bill claim form facilitates transparency between payors and potential healthcare providers to ensure, for example, that the prices of services are at the CPT® code level with competitive bidding (considering, e.g., cost, speed to care, location/distance, service types, language spoken, treatment protocols, and other factors).

In flow diagram 400, a workflow transactions engine, e.g., workflow transactions engine 210, is configured to generate a pre-bill claim form based on one or more medical classification codes. For example, a script indicating the one or more medical classification codes (e.g., ICD codes, CPT® codes, etc.) is obtained at step 402. If a bill builder template exists for one or more medical classification codes within an accessible storage device at step 404, a pre-bill claim form is generated from the template at step 406, wherein the patient's location (e.g., based on the patient's zip code) is used to ensure that the pre-bill claim form complies with local/state jurisdictional guidelines. If a bill builder template does not exist for the medical classification codes within the accessible storage device, an Official Disability Guidelines (ODG) API call is instantiated at step 408. If the medical classification codes are found at step 410, filters relevant to the medical classification codes are applied to a bill builder template at step 412 and the pre-bill claim form is generated from the template at step 406. For example, the pre-bill claim form may be generated using a statistical or machine learning technique including one or more of a data mining, predictive modelling, and deep learning technique as described above. The task is referred to an operations queue at step 414 if the medical classification codes are not found.

After the pre-bill claim form is generated, a clearinghouse API call is instantiated at step 416. For example, systems may use a combination of internal capabilities or source a single combination of third part clearinghouses to ensure complete jurisdictional coverage. Bill conditions such as general formatting, validation of fee schedule coding matches diagnostic coding applicability for codes may be jointly submitted. If the clearinghouse API is successful at step 418, it is determined if any rules (e.g.,) are triggered at step 420. For example, a rule may be triggered for medical classification codes that are not allowed for reimbursement, or other types of rule violations. For example, the types of rules may include bill knock out rules (e.g., formatting, boundary and limit constraints, confirmation of fee schedules and/or usual and customary rates, and legally recognized claim coding guidelines). If the clearinghouse API is not successful at step 418, the case is referred to an admin queue for further attention at step 422. If no rules are triggered at step 420, the one or more medical classification codes are used for selecting healthcare providers to participate in a bid auction at step 424 and the method proceeds to an auction engine process at step 426. If rules are triggered at step 420, medical classification codes that are not allowed are removed at step 428. The filtered one or more medical classification codes are then used for selecting healthcare providers to participate in a bid auction at step 430, and the method proceeds to the auction engine process at step 426. If no codes in the pre-bill claim form are allowed after the filtering at step 428, the case is referred to an operations queue for further attention at step 432.

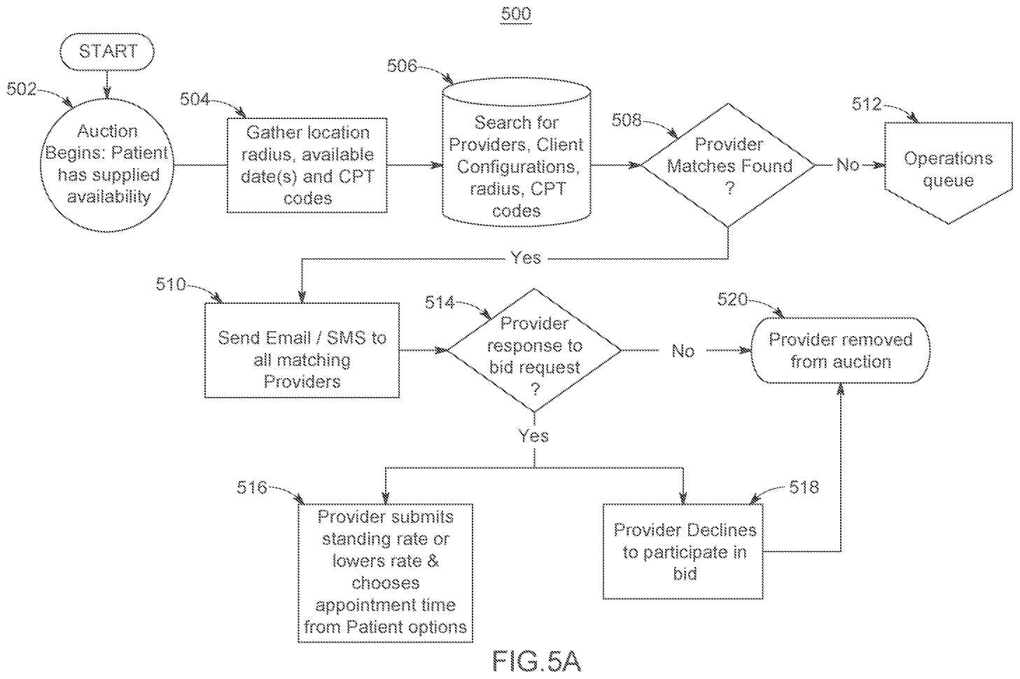

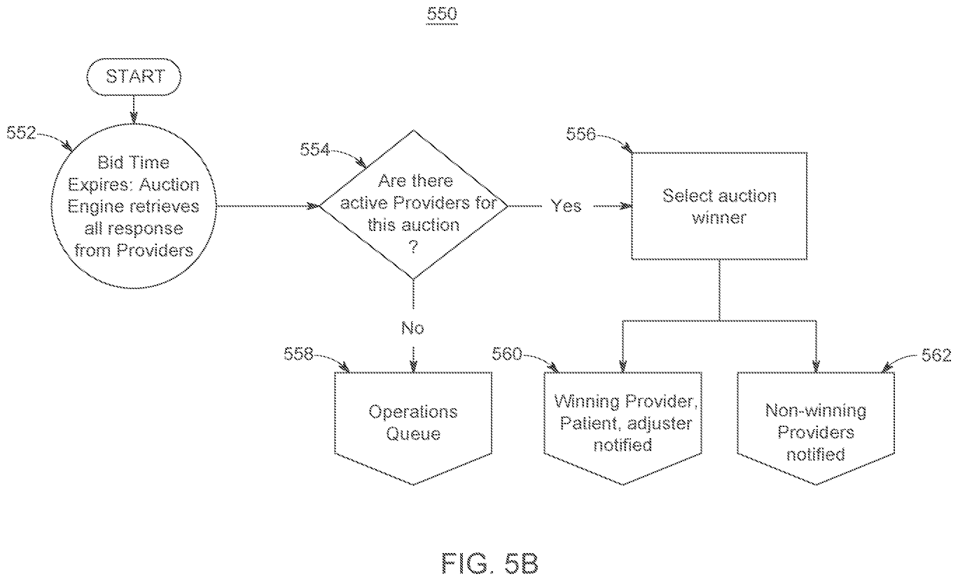

FIG. 5A illustrates a flow diagram of example operations for selecting healthcare providers from among the plurality of healthcare providers to participate in a bid auction in accordance with an embodiment. In flow diagram 500, an auction engine, e.g., auction engine 220, is configured to select healthcare providers from among the plurality of healthcare providers to participate in a bid auction. For example, a bid auction begins at step 502 based on the patient's availability obtained in a request to procure healthcare services as described in step 302 above. The information regarding the patient's availability may include at least one of a time and date parameter, and geographical information related to the patient such as, for example, a radius around a geographical location or a distance from an address or location of the patient. This information, along with one or more medical classification codes related to the healthcare services requested are used at step 504 to search for potential healthcare service providers at step 506. For example, provider subscription profiles corresponding to each of a plurality of potential healthcare providers may be generated based on subscriber information and provider subscription criteria. The provider subscription profiles may include, for example, provider data related to one or more of demographic information, financial information, location information, practice information, procedural codes, and pricing information. In some embodiments, the provider subscription criteria may be related to workers' compensation healthcare services, managed care plans, or other group healthcare services. If one or more healthcare provider matches are found at step 508, a bid invitation, e.g., an email or Short Message Service/Multi-media Messaging Service (SMS/MMS) message, is sent to the healthcare providers selected to participate at step 510. If no healthcare provider matches are found at step 508, the result is sent to an operations queue at step 512. If an invited healthcare provider responds to the bid invitation at step 514, the healthcare provider response, as illustrated at step 516, may include a standing or customized (e.g., lower) rate and a selection of an appointment time based on the patient's availability options. Alternatively, as illustrated at step 518, the healthcare provider may decline to submit a bid. Further, if an invited healthcare provider does not respond to the bid invitation at step 514, the unresponsive healthcare provider is removed from the bid auction at step 520.

In an embodiment, bid auction invitations may be generated by processing the provider subscription profiles using a selection algorithm as described above. For example, the selection algorithm may be operable to assign a bid invitation ranking to each provider subscription profile based on the request and ranking parameters, and select healthcare providers from among the plurality of healthcare providers to participate in a bid auction based on the bid invitation rankings. The ranking parameters may be derived from the provider information based on one or more of the following: services provided, location, government regulations, client/payor preferences, auction configuration parameters, scheduling availability, quality metrics, and behavior metrics. In an exemplary embodiment, the ranking parameters may be derived using a statistical or machine learning technique including one or more of a data mining, predictive modelling, and deep learning technique, and a selection model trained using training data derived from, e.g., training provider subscription profiles corresponding to the one or more healthcare providers, may be instantiated to determine ranking parameters, a “next best action”, and/or other “actionable insights” related to selecting healthcare providers to participate in the bid auction.

FIG. 5B illustrates a flow diagram of example operations for automatically conducting a bid auction among healthcare providers selected to participate in accordance with an embodiment. In flow diagram 550, an auction engine, e.g., auction engine 220, is further configured to conduct a bid auction among the healthcare providers selected to participate. For example, bid responses (if any) from the healthcare providers selected to participate are received upon expiration of the time limit for the bid auction at step 552. If, at step 554, bid responses are received from healthcare providers selected to participate, an auction winner is selected at step 556. Otherwise, the auction result is referred to an operations queue at step 558. For example, the auction engine may be configured using the selection model described above and one or more medical classification codes related to the healthcare services to automatically conduct a bid auction among the healthcare providers selected to participate, wherein the bid auction results in a selection of at least one of the plurality of healthcare providers to provide the healthcare services.

At step 560, the at least one of the plurality of healthcare providers selected to provide the healthcare services is notified of the bid auction result. Likewise, the healthcare providers not selected to provide the healthcare services are notified at step 562.

It should be noted that the bid auction described herein contemplates an alternative to long-term fixed pricing contracts for healthcare services. Rather, in the various embodiments each individual script may establish its own spot price contract based on the amounts bid, or the “Buy it Now” prices established by the healthcare providers selected to participate. Further, in preferred embodiments, the reimbursement amount for any individual script does not influence any other scripts awarded to a healthcare provider.

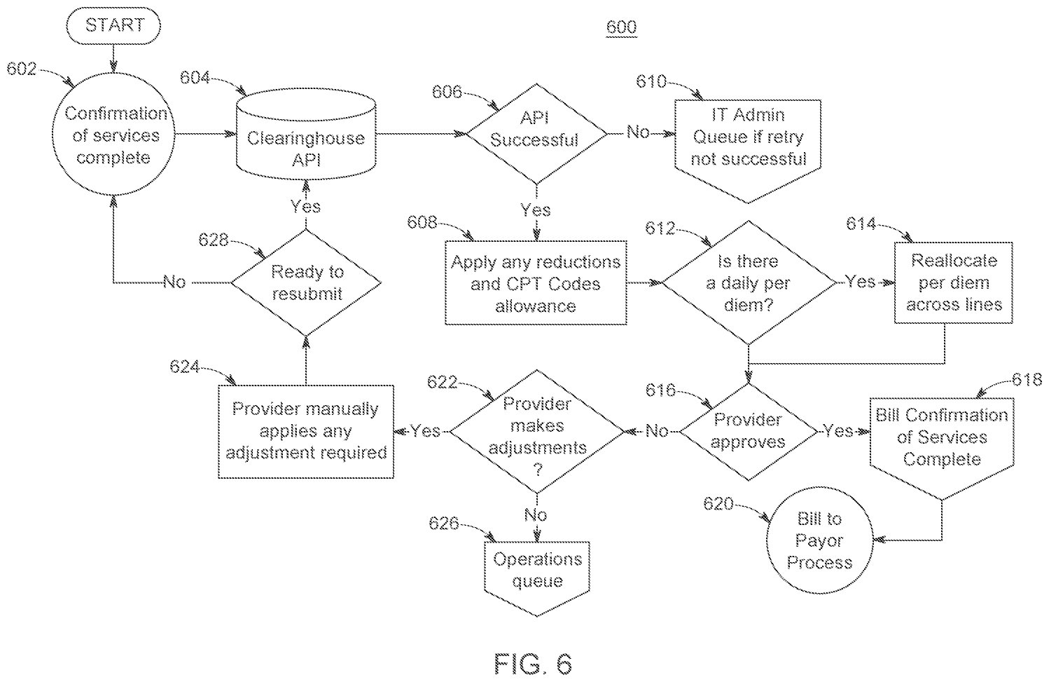

FIG. 6 illustrates a flow diagram of example operations for generating an invoice for the healthcare services performed in accordance with an embodiment. In flow diagram 600, a workflow transaction engine, e.g., workflow transaction engine 210, is configured to generate an invoice for the healthcare services performed. For example, upon confirmation that the healthcare services have been performed at step 602, a clearinghouse API call is instantiated at step 604. The API receives a data payload submission from the platform, e.g., healthcare procurement platform 108, and tests to ensure that the content, including time, date and jurisdictional constraints, is appropriate and accurate, and the formatting of the bill meets jurisdictional standards. If the clearinghouse API is successful at step 606, medical classification code reductions and allowances are applied at step 608. Clearinghouse API success is achieved when, in a roundtrip call-out and response from a third-party API, the API sends validation that the transaction payload is acceptable and is formatted in the correct configuration. If the clearinghouse API is not successful at step 606, the case is referred to an admin queue for further attention at step 610.

After the medical classification code reductions and allowances are applied at step 608, it is determined if a daily per diem limit applies to the invoice at step 612. The per diem limit is the maximum amount billable based on a single day of treatment. If the sum of the single day of treatments exceeds the per diem amount, the individual line-item amounts are proportionally reduced so the daily total is equal to the per diem limit. If a daily per diem does apply, the per diem is allocated across lines at step 614. If a daily per diem does not apply at step 612, and the healthcare provider approves of the pre-bill at step 616, the bill confirmation process is complete at step 618 and the method proceeds to operations for invoicing the payor at step 620.

Returning to step 616, if the healthcare provider does not approve of the pre-bill, it is determined if the provider would like to make manual adjustments to the pre-bill at step 622. If the provider would like to make manual adjustments, the provider is enabled to make the adjustments at step 624. While the pre-bill process described herein is uniquely accurate, there may situations where the provider based on assessment, review and consultation with the patient wants to alter the course of care from what was preapproved. These manual adjustments ensure that what is actually billed matches the exact services that were administered to the patient. However, if the healthcare provider does not approve of the pre-bill and does not have any manual adjustments to make, the case is referred to an admin queue for further attention at step 626.

Upon receipt of the manual adjustments, it is determined if the pre-bill is ready for resubmission at step 628. If so, the method returns to the clearinghouse API call at step 604. If the pre-bill is not ready for resubmission at step 628, the method returns to confirming whether the healthcare services have been performed at step 602.

Electronic Funds Transfer (EFT), Automated Clearing House (ACH), PayPal® or another known electronic payment method may be offered or automatically initiated to complete payment transactions. Alternatively, check payments may be issued upon request. In exemplary embodiments, invoices to payors will be issued to comply with state jurisdictional guidelines and will include an explanation of the reimbursement amount.

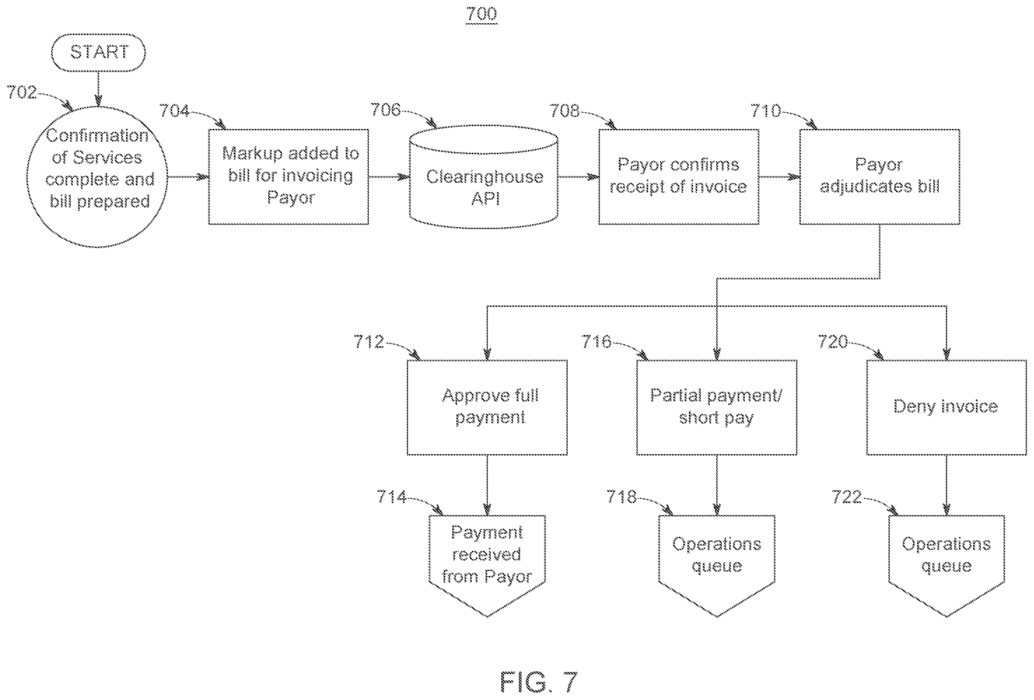

FIG. 7 illustrates a flow diagram of example operations for invoicing a payor for the healthcare services performed in accordance with an embodiment. In flow diagram 700, a billing engine, e.g., billing engine 240, is further configured to invoice a payor for the healthcare services performed. For example, at step 702, upon confirmation that the healthcare services have been performed and the invoice for the service has been generated and approved as described above, a service mark-up may be added to the invoice by, for example, an administrator/operator of the PaaS at step 704. This markup may be jointly agreed between the payor and the marketplace administrator/operator based on one or more volume and economic factors. After the mark-up is applied, a clearinghouse API call is instantiated at step 706, which includes an action, e.g., communicating the invoice to the payor. Upon confirmation that the payor has received the invoice at step 708, the payor adjudicates the invoice at step 710. For example, the payor may approve the invoice for payment at step 712 and submit a full payment at step 714. Alternatively, the payor may approve a partial payment for the invoice at step 716, and the case may be referred to an admin queue for further attention at step 718. In another alternative, the payor may deny payment of the invoice at step 720, and the case may be referred to an admin queue for further attention at step 722.

In an embodiment, operations for invoicing a payor may include configuring and/or instantiating a computing device or network platform to take an action, such as generating the invoice to a payor of the healthcare services. The action may also include one or more of generating an alert notification, and automatically initiating an Electronic Funds Transfer (EFT), Automated Clearing House (ACH) or other electronic payment method.

Thus, presented herein are systems and methods for managing the procurement of healthcare services. Traditionally, such management has required using non-integrated, manual processes for connecting payors, healthcare providers and patients, however, the systems and methods herein can be used for healthcare providers to identify and bid on new patient scripts from multiple payor sources leveraging system integrations, and data backed automated decision making in real-time. This automated decision making may include, for example, selecting auction participants and conducting bid auctions based on continuous recalculation of weighted healthcare provider and payor data values and selection criteria (e.g., speed to care, location, quality, price, treatment protocols, etc.) during run-time operations. The systems and methods herein may further enable transparency between patients, healthcare providers and payors by enabling adjustments or dynamic recalculations within algorithms used for selecting auction participants and conducting bid auctions based on past or present payer preferences, market conditions, engagement level, or various real-time insights derived from predictive analytics. Further, systems and methods facilitate expedited billing of such services.

Systems, apparatus, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computers and interact via a network. The client-server relationship may be defined and controlled by computer programs running on the respective client and server computers.

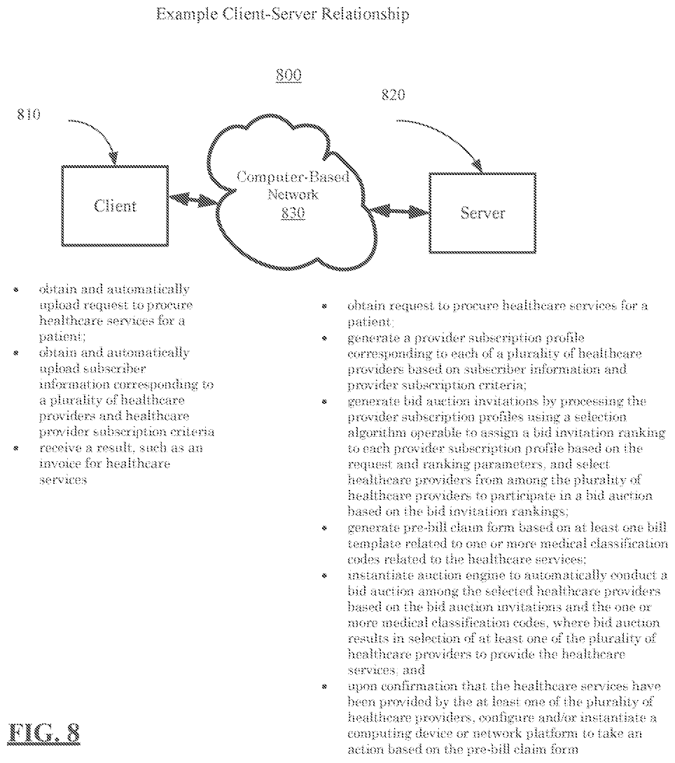

A high-level block diagram of an exemplary client-server relationship that may be used to implement systems, apparatus and methods described herein is illustrated in FIG. 8. Client-server relationship 800 comprises client 810 in communication with server 820 via computer-based network 830 and illustrates one possible division for procuring healthcare services between client 810 and server 820. For example, client 810, in accordance with the various embodiments described above, may obtain and automatically upload request to procure healthcare services for a patient; obtain and automatically upload subscriber information corresponding to a plurality of healthcare providers and healthcare provider subscription criteria and receive a result, such as an invoice for healthcare services. Server 820 may obtain request to procure healthcare services for a patient; generate a provider subscription profile corresponding to each of a plurality of healthcare providers based on subscriber information and provider subscription criteria; generate bid auction invitations by processing the provider subscription profiles using a selection algorithm operable to assign a bid invitation ranking to each provider subscription profile based on the request and ranking parameters, and select healthcare providers from among the plurality of healthcare providers to participate in a bid auction based on the bid invitation rankings; generate pre-bill claim form based on at least one bill template related to one or more medical classification codes related to the healthcare services; instantiate an auction engine to automatically conduct a bid auction among the selected healthcare providers based on the bid auction invitations and the one or more medical classification codes, wherein the bid auction results in a selection of at least one of the plurality of healthcare providers to provide the healthcare services; and upon confirmation that the healthcare services have been provided by the at least one of the plurality of healthcare providers, configure and/or instantiate a computing device or network platform to take an action based on the pre-bill claim form.

One skilled in the art will appreciate that the exemplary client-server relationship illustrated in FIG. 8 is only one of many client-server relationships that are possible for implementing the systems, apparatus, and methods described herein. As such, the client-server relationship illustrated in FIG. 8 should not, in any way, be construed as limiting. Examples of client devices 810 can include cellular smartphones, kiosks, personal data assistants, tablets, robots, vehicles, web cameras, or other types of computing devices.

Systems, apparatus, and methods described herein may be implemented using a computer program product tangibly embodied in an information carrier, e.g., in a non-transitory machine-readable storage device, for execution by a programmable processor; and the method steps described herein, including one or more of the steps of FIGS. 3-7, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

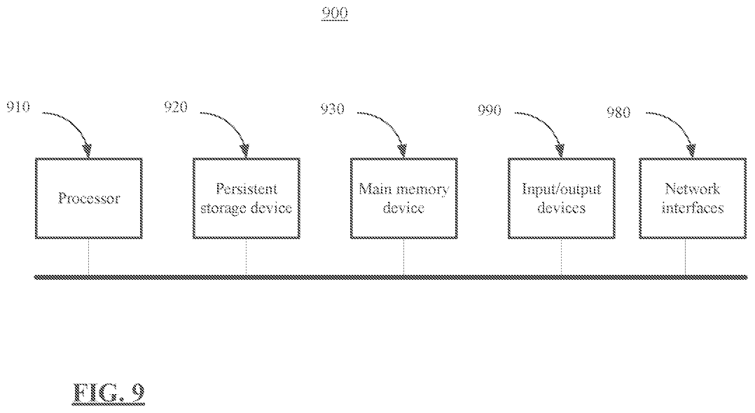

A high-level block diagram of an exemplary apparatus that may be used to implement systems, apparatus and methods described herein is illustrated in FIG. 9. Apparatus 900 comprises a processor 910 operatively coupled to a persistent storage device 920 and a main memory device 930. Processor 910 controls the overall operation of apparatus 900 by executing computer program instructions that define such operations. The computer program instructions may be stored in persistent storage device 920, or other computer-readable medium, and loaded into main memory device 930 when execution of the computer program instructions is desired. For example, workflow transaction engine 210 and auction engine 220 may comprise one or more components of apparatus 900. Thus, the method steps of FIGS. 3-7 can be defined by the computer program instructions stored in main memory device 930 and/or persistent storage device 920 and controlled by processor 910 executing the computer program instructions. For example, the computer program instructions can be implemented as computer executable code programmed by one skilled in the art to perform an algorithm defined by the method steps of FIGS. 3-7. Accordingly, by executing the computer program instructions, the processor 910 executes an algorithm defined by the method steps of FIGS. 3-7. Apparatus 900 also includes one or more network interfaces 980 for communicating with other devices via a network. Apparatus 900 may also include one or more input/output devices 990 that enable user interaction with apparatus 900 (e.g., display, keyboard, mouse, speakers, buttons, etc.).

Processor 910 may include both general and special purpose microprocessors and may be the sole processor or one of multiple processors of apparatus 900. Processor 910 may comprise one or more central processing units (CPUs), and one or more graphics processing units (GPUs), which, for example, may work separately from and/or multi-task with one or more CPUs to accelerate processing, e.g., for various image processing applications described herein. Processor 910, persistent storage device 920, and/or main memory device 930 may include, be supplemented by, or incorporated in, one or more application-specific integrated circuits (ASICs) and/or one or more field programmable gate arrays (FPGAs).

Persistent storage device 920 and main memory device 930 each comprise a tangible non-transitory computer readable storage medium. Persistent storage device 920, and main memory device 930, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 990 may include peripherals, such as a printer, scanner, display screen, etc. For example, input/output devices 990 may include a display device such as a cathode ray tube (CRT), plasma or liquid crystal display (LCD) monitor for displaying information to a user, a keyboard, and a pointing device such as a mouse or a trackball by which the user can provide input to apparatus 900.

Any or all of the systems and apparatuses discussed herein, including workflow transaction engine 210 and auction engine 220 may be performed by, and/or incorporated in, an apparatus such as apparatus 900. Further, apparatus 900 may utilize one or more neural networks or other deep-learning techniques to perform workflow transaction engine 210 and auction engine 220 or other systems or apparatuses discussed herein.

One skilled in the art will recognize that an implementation of an actual computer or computer system may have other structures and may contain other components as well, and that FIG. 9 is a high-level representation of some of the components of such a computer for illustrative purposes.

The foregoing specification is to be understood as being in every respect illustrative and exemplary, but not restrictive, and the scope of the invention disclosed herein is not to be determined from the specification, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A computer-based system for procuring healthcare services, the system comprising:
a non-transitory, computer readable memory storing software instructions; and
at least one processor coupled with the memory operating as a workflow transaction engine and, upon execution of the software instructions, configured as a special-purpose processor to:
obtain a request to procure healthcare services for a patient;
generate a provider subscription profile corresponding to each of a plurality of healthcare providers based on subscriber information and provider subscription criteria;
generate bid auction invitations by processing the provider subscription profiles using a selection algorithm operable to assign a bid invitation ranking to each provider subscription profile based on the request and ranking parameters, and select healthcare providers from among the plurality of healthcare providers to participate in a bid auction based on the bid invitation rankings;
generate a pre-bill claim form based on at least one bill template related to one or more medical classification codes related to the healthcare services;
instantiate an auction engine to automatically conduct a bid auction among the selected healthcare providers based on the bid auction invitations and the one or more medical classification codes, wherein the bid auction results in a selection of at least one of the plurality of healthcare providers to provide the healthcare services; and
upon confirmation that the healthcare services have been provided by the at least one of the plurality of healthcare providers, configure a computing device or network platform to take an action based on the pre-bill claim form.

2. The system of claim 1, wherein the healthcare services comprise one or more of medical, pharmaceutical, prescription, psychiatric/mental health, and dental services.

3. The system of claim 1, wherein the patient is an injured worker and the healthcare services are workers' compensation healthcare services.

4. The system of claim 1, wherein the patient's healthcare insurance coverage is provided via at least one of an employer-sponsored group health benefits plan, and a government-sponsored health plan.

5. The system of claim 1, wherein the patient is an individual consumer of healthcare services.

6. The system of claim 1, wherein the request comprises information regarding the patient's availability including at least one of a time and date parameter.

7. The system of claim 1, wherein the request comprises geographical information related to the patient.

8. The system of claim 7, wherein the geographical information includes a radius around a geographical location.

9. The system of claim 7, wherein the geographical information includes a distance from an address or location of the patient.

10. The system of claim 1, wherein the provider subscription criteria are related to workers' compensation healthcare services.

11. The system of claim 1, wherein the provider subscription criteria include parameters based on one or more of the following: provider demographic information, provider financial information, provider location information, provider practice information, provider procedural codes, and provider pricing information.

12. The system of claim 1, wherein the provider subscription profiles include provider data related to one or more of the following: demographic information, financial information, location information, practice information, procedural codes, and pricing information.

13. The system of claim 1, wherein the one or more medical classification codes comprise Current Procedural Terminology® (CPT®) codes or codes conforming to another nomenclature for medical procedures or supplies.

14. The system of claim 1, wherein the one or more medical classification codes comprise International Classification of Diseases, Tenth Revision, Clinical Modification (ICD-10-CM) codes or codes conforming to another nomenclature for disease classification.

15. The system of claim 1, wherein the special-purpose processor is further configured to generate the pre-bill claim form using a statistical or machine learning technique including one or more of a data mining, predictive modelling, deep learning, or other artificial intelligence technique.

16. The system of claim 1, wherein the pre-bill claim form is a CMS-1500 claim form or a successor CMS claim form.

17. The system of claim 1, wherein the bid auction invitations comprise the pre-bill claim form.

18. The system of claim 1, wherein the pre-bill claim form comprises an individual bid contract.

19. The system of claim 1, wherein the special-purpose processor is further configured to derive the ranking parameters from the provider information based on one or more of the following: services provided, location, government regulations, client/payor preferences, auction configuration parameters, scheduling availability, quality metrics, and behavior metrics.

20. The system of claim 1, wherein the special-purpose processor is further configured to derive the ranking parameters using a statistical or machine learning technique including one or more of a data mining, predictive modelling, and deep learning technique.

21. The system of claim 20, wherein the special-purpose processor is further configured to train a selection model using the provider subscription profiles corresponding to the one or more healthcare providers.

22. The system of claim 21, wherein the auction engine is configured using the trained selection model to automatically conduct the auction among the selected healthcare providers.

23. The system of claim 1, wherein the special-purpose processor is further configured to instantiate the computing device or network platform to take the action based on the pre-bill claim form.

24. The system of claim 1, wherein the action comprises one or more of generating invoice to a payor of the healthcare services, generating an alert notification, and automatically initiating an Electronic Funds Transfer (EFT), Automated Clearing House (ACH) or other electronic payment method.

25. A computer-based system for procuring healthcare services, the system comprising:
a non-transitory, computer readable memory storing software instructions; and
at least one processor coupled with the memory operating as a workflow transaction engine and, upon execution of the software instructions, configured as a special-purpose processor to:
obtain a request to procure healthcare services for a patient;
generate a provider subscription profile corresponding to each of a plurality of healthcare providers based on subscriber information and provider subscription criteria;
generate bid auction invitations by processing the provider subscription profiles using a selection algorithm operable to assign a bid invitation ranking to each provider subscription profile based on the request and ranking parameters, and select healthcare providers from among the plurality of healthcare providers to participate in a bid auction based on the bid invitation rankings;
generate a pre-bill claim form based on at least one bill template related to one or more medical classification codes related to the healthcare services;
instantiate an auction engine to automatically conduct a bid auction among the selected healthcare providers based on the bid auction invitations and the one or more medical classification codes, wherein the bid auction results in a selection of at least one of the plurality of healthcare providers to provide the healthcare services; and
upon confirmation that the healthcare services have been provided by the at least one of the plurality of healthcare providers, configure a computing device or network platform to take an action based on the pre-bill claim form.

26. A non-transitory computer-readable medium having computer instructions stored thereon for procuring healthcare services, which, when executed by a processor, cause the processor to perform one or more steps comprising:
obtaining a request to procure healthcare services for a patient;
generating a provider subscription profile corresponding to each of a plurality of healthcare providers based on subscriber information and provider subscription criteria;
generating bid auction invitations by processing the provider subscription profiles using a selection algorithm operable to assign a bid invitation ranking to each provider subscription profile based on the request and ranking parameters, and select healthcare providers from among the plurality of healthcare providers to participate in a bid auction based on the bid invitation rankings;
generating a pre-bill claim form based on at least one bill template related to one or more medical classification codes related to the healthcare services;
instantiating an auction engine to automatically conduct a bid auction among the selected healthcare providers based on the bid auction invitations and the one or more medical classification codes, wherein the bid auction results in a selection of at least one of the plurality of healthcare providers to provide the healthcare services; and
upon confirmation that the healthcare services have been provided by the at least one of the plurality of healthcare providers, configuring a computing device or network platform to take an action based on the pre-bill claim form.
